(19) United States
(12) Patent Application Publication
(10) Pub. No.: US 2013/0203110 A1
(43) Pub. Date: Aug. 8, 2013

(54) SYSTEM FOR THE IN VITRO TRANSCRIPTION AND TRANSLATION OF MEMBRANE PROTEINS

(71) Applicants: Klaus-Stefan Drese, Mainz (DE); Daniel Latta, Mainz (DE); Angelika Murr, Mainz (DE); Marion Ritzi-Lehnert, Langen (DE); Eva-Kathrin Sinner, Gau-Algesheim (DE)

(72) Inventors: Klaus-Stefan Drese, Mainz (DE); Daniel Latta, Mainz (DE); Angelika Murr, Mainz (DE); Marion Ritzi-Lehnert, Langen (DE); Eva-Kathrin Sinner, Gau-Algesheim (DE)

(73) Assignee: INSTITUT FUER MIKROTECHNIK MAINZ GMBH, Mainz (DE)

(21) Appl. No.: 13/640,451

(22) PCT Filed: Apr. 20, 2010
(86) PCT No.: PCT/EP2010/055206

§ 371 Date: Dec. 26, 2012

(51) Int. Cl. C12P 21/00

(52) U.S. Cl. CPC C12P 21/00
USPC 435/68.1; 435/289.1

(57) ABSTRACT

System for the in vitro transcription and translation of membrane proteins comprising i) a micro-fluidic chip having at least one micro-fluidic reaction chamber and micro-fluidic channels to allow fluid to flow through the chip and into and from the at least one reaction chamber, ii) the at least one micro-fluidic reaction chamber being provided with at least one electrode base plate of conductive or semi-conductive material, and iii) lipid vesicles or a lipid membrane being bound or tethered to the at least one electrode base plate either directly or through spacer molecules.

The present invention relates to a system for the in vitro transcription and translation of membrane proteins into lipid vesicles or lipid membranes.

### BACKGROUND OF THE INVENTION

The investigation of membrane receptor/ligand interaction is a prerequisite for understanding complex biological pathways involving cell membranes. To perform such investigations, efficient and reproducible in vitro assay systems are required to characterize specific receptor/ligand binding interaction in isolation from other interactions, in which the receptor may be engaged in natural environments.

Therefore, model systems for biological membranes have been developed such as liposomes, planar black lipid membranes (BLMs) as well as solid-supported membranes such as solid-supported lipid bilayers and tethered lipid bilayers. Tethered lipid membranes (tBLMs) are solid-supported lipid films with hydrophilic spacer groups such as peptide, polyethylene glycol or sugar groups, tethered covalently to a support. To incorporate membrane protein into such model membrane systems, isolation of membrane proteins and reconstitution into the membrane system has been necessary so far. Thus, for example, forming of a phospholipid monolayer on a solid support and subsequently subjecting this monolayer to lipid vesicles containing acetylcholine receptor (AChR) leads to the incorporation of the acetylcholine receptor into a bilipid membrane, wherein the second layer is formed from lipids contained in the lipid vesicle (E. K. Schmidt et al., Biosensors and Bioelectronics 13 (1998) 585-591). R. Naumann et al. (Biosensors and Bioelectronics 14 (1999) 651-662 describe the incorporation of cytochrome c oxidase in functionally active form into a peptide-tethered lipid membrane. Liposomes comprising phosphatidylcholine are spread on a thiopeptide-lipid monolayer to form a peptide-tethered lipid bilayer membrane. This membrane is then incubated with isolated cytochrome c oxidase.

A similar approach has been reported for incorporation of integrins into artificial planar lipid membranes (E. K. Sinner, Analytical Biochemistry 333 (2004) 216-224). In this approach, integrins were incorporated into a lipid-functionalized peptide layer by vesicle spreading. Also with this approach membrane protein-containing vesicles had to be prepared first, requiring preparation and isolation of the membrane protein.

A problem in the case of the hitherto used manufacturing methods was that the membrane proteins had to be isolated first. The native activity of the proteins was often lost thereby. Further, incorporation into the membranes, e.g. in the case of vesicle spreading, was often effected randomly, however, not directed. This led to non-optimal test systems, in which the interaction and orientation of the proteins with and in the membrane as well as its effect on interactions with ligands cannot be investigated.

To solve this problem, WO 2007/048459 provides an improved method for the preparation of membranes having membrane proteins incorporated therein, in particular a method, wherein the membrane proteins do not have to be isolated first. The method of WO 2007/048459 for the preparation of membrane proteins uses a cell-free in vitro transcription and translation system in the presence of a membrane bound or tethered on a gold substrate. The method allows the translated proteins to be integrated into the membrane in their native functional form, without the membrane proteins having to be isolated first. Further, the membranes produced according to this method are described to have high stability, since due to the use of cell-free expression systems, for example, no protein-degrading proteases are present in the system.

One problem of the prior art methods disclosed in WO 2007/048459 is that the known in vitro transcription and translation systems allow only small amounts of proteins to be synthesized. Large scale reactions can result in low yields and ineffective reaction courses, probably due to inhomogeneous temperature distribution and temperature gradients within the reaction vessel.

Other systems, using protein synthesis in cell culture, encompass other severe problems or disadvantages, for example, production of not only one ore more of the desired membrane proteins, but also a number of other cellular proteins, difficulties to quantify the translated proteins, protein degradation and digestion problems, and irreproducibility due to changes of the cultured cells during lifetime and in response to environmental factors.

### OBJECT OF THE INVENTION

Accordingly, it is an object of the invention to provide a system for the in vitro transcription and translation of membrane proteins that allows for a more reproducible and larger scale production as well as a larger spectrum of the membrane proteins compared to the prior art.

### DESCRIPTION OF THE INVENTION

The problem of the invention is solved by a system for the in vitro transcription and translation of membrane proteins comprising

i) a micro-fluidic chip having at least one micro-fluidic reaction chamber and micro-fluidic channels to allow fluid to flow through the chip and into and from the at least one reaction chamber,
ii) the at least one micro-fluidic reaction chamber being provided with at least one electrode base plate of conductive or semi-conductive material,
iii) lipid vesicles or a lipid membrane being bound or tethered to the at least one electrode base plate either directly or through spacer molecules.

It is presumed that the in vitro transcription and translation of membrane proteins with a cell-free expression system in the presence of a membrane facilitates or stabilises the expression since the membrane acts as a quasi-substitute for the endoplasmatic reticulum. Cell-free expression systems have been used to express soluble proteins in aqueous systems, whereby in many cases no natively active proteins but denatured proteins, e.g. in the form of inclusion bodies, are obtained.

Kits for carrying out in vitro transcription and translation reactions are commercially available. The in vitro transcription and translation reactions are usually carried out in a reaction volume of about 25 to 50 μl in standard reaction vials, such as plastic Eppendorf tubes or the like, according to the manufacturers instructions. This obviously works well for many soluble proteins. However, particularly for membrane proteins the efficiency of the in vitro transcription and translation reactions under these standard conditions has been found to be unsatisfying.

It has now been found by the present inventors that the dimensions and geometry of the reaction space where the in vitro transcription and translation of the membrane proteins is carried out plays an important role for the transcription and translation efficiency.

The efficiency of the in vitro transcription and translation reactions could surprisingly be drastically improved by carrying out the reaction in a micro-fluidic reaction chamber of a micro-fluidic chip. Micro-fluidic chips for carrying out chemical and biological reactions are as such well known. However, it has never been shown to produce membrane proteins in a micro fluidic chip. Also, it could not be expected to improve the efficiency of the in vitro transcription and translation reactions to produce membrane proteins since such reactions are already under standard conditions carried out in very small volumes, following the usual protocols of the manufacturers instructions or scientific handbooks. It was therefore very surprising that carrying out the in vitro transcription and translation reactions in a device having the dimensions and geometry of a micro fluidic chip compared to a small volume standard reaction vessel, such as an Eppendorf tube, resulted in an increase in the reaction efficiency.

It has further been surprisingly found by the present inventors that the efficiency of the in vitro transcription and translation reactions can be improved by observing a minimum ratio of the surface (A) by the volume (V) of the reaction chamber or the fluid in the reaction chamber. The inventors assume, but do not want to be bound or restricted by this theory, that the dimensions and geometry leading to the surface (A) by volume (V) ratio (A/V) of the reaction chamber result in an improvement in the thermal homogeneity within the reaction solution (fluid). This thermal homogeneity is assumed to be the or at least one reason for the improved efficiency of the in vitro transcription and translation of the membrane proteins according to the present invention.

Therefore, in a preferred embodiment of the present invention the at least one micro-fluidic reaction chamber has dimensions and geometry to provide for a surface (A) by volume (V) ratio (A/V) of the fluid in the reaction chamber of at least 1 mm⁻¹. In a more preferred embodiment the at least one micro-fluidic reaction chamber has dimensions and geometry to provide for a surface (A) by volume (V) ratio (A/V) of the fluid in the reaction chamber of at least 3 mm⁻¹, more preferably at least 5 mm⁻¹ or at least 10 mm⁻¹.

In another preferred embodiment of the present invention the at least one micro-fluidic reaction chamber has a cross sectional area over its entire length of 2×10⁻³ μm² to 3×10⁶ μm². Even more preferred the at least one micro-fluidic reaction chamber has a cross sectional area over its entire length of 1 μm² to 1×10⁶ μm², more preferably 1×10³ μm² to 0.5×10⁶ μm². If the cross sectional area becomes to small, the passage of the largest particles of the in vitro transcription and translation constituents through the reaction chamber may be inhibited. If the cross sectional area becomes to large, the thermal homogeneity may be impaired.

In another preferred embodiment of the present invention the at least one electrode base plate consists of or has a surface consisting of a metal, metal oxide, polymeric materials, glass, field effect transistors, or indium tin oxide (ITO). Most preferably the electrode base plate consists of gold. The electrode base plate further provides electrical connectors for the connection of the electrode base plate to a measuring device to conduct electrochemical measurements or the like.

In another preferred embodiment of the present invention the lipid vesicles or the lipid membrane are/is bound or tethered to the at least one electrode base plate through spacer molecules, preferably the spacer molecules being selected from the group consisting of human serum albumine molecules (HAS), bovine serum albumine molecules (BSA) or cationic bovine serum albumine molecules (cBSA), poly-peptide or oligo-peptide molecules, PEG, sugar molecules, silane molecules, silane/thiol molecules, or polymer molecules.

In another preferred embodiment of the present invention hydrophilic spacer molecules are used. If peptides are used as hydrophilic spacer molecules the peptide spacer molecules preferably have a length of 3 to 100, preferably 4 to 30, more preferably 5 to 25 amino acids. The chosen sequences preferably have a cysteine residue on one end. When using a gold surface, a monomolecular peptide layer can be obtained by self-assembly caused by strong gold-sulfur interaction of the preferred terminal N-cysteine moiety. The 19-mer peptide CSRARKQAASI KVAVSADR (P19) derived from the alpha-laminin subunit has proven particularly useful.

The lipid vesicles or the lipid membrane of the present invention can be of any suitable material that has vesicle or membrane properties. However, it is preferred that the lipid vesicles or the lipid membrane of the present invention are/is synthetic or comprise/s natural membrane components, synthetically produced lipids, phospholipids, preferably 1,2-Dimyristoyl-sn-glycero-3-phosphoethanolamine (DMPE) or phosphatidylcholine. It is further preferred if the lipid vesicles or the lipid membrane comprise/s a lipid bilayer.

In another preferred embodiment of the present invention the membrane used is a synthetic membrane, i.e. not a membrane of natural origin. This is advantageous because reproducible model systems with specific desired properties can be obtained thereby. Potential undesired interactions, which might be caused by components of natural membranes, can be excluded. The membrane used according to the invention, therefore, preferably consists of synthetically produced lipids, in particular, phospholipids. However, it is also possible to employ natural membranes or fragments of natural membranes, e.g. microsomes.

For subsequent examination and investigation of the system, the membrane protein can be coupled with a tag. The tag preferably is selected from epitopes which allow binding of a specific antibody thereto. Examples of suitable tags are VSV (vesicular stomatitis virus glycoprotein), His tag, Strep tag, Flag tag, intein tag or GST tag. However, it is also possible to couple to the membrane protein a partner of a high affinity binding pair such as biotin or avidin or a label such as an enzyme label or a fluorescent label which allows direct determination of the membrane protein.

The tag then can be used to couple a detectable group, such as a fluorescent group, to the membrane protein. Examination of the membrane and, in particular of the presence and activity of membrane proteins, for example, can be performed by Surface Plasmon Resonance Spectroscopy or Surface Plasmon-enhanced Fluorescence Spectroscopy (SPFS). These methods allow to monitor the assembling of lipid membranes and binding interactions of incorporated membrane proteins in real time. These methods also allow detection and monitoring of very few protein molecules within a membrane such as 10 to 10000, in particular, 100 to 1000 protein molecules.

The present invention also includes a process for the in vitro transcription and translation of membrane proteins comprising

i) providing a system according to any of the preceding claims,
ii) applying a cell-free expression system and a nucleic acid coding for the membrane proteins to the lipid vesicles or the lipid membrane in the at least one micro-fluidic reaction chamber, and
iii) expression of the membrane proteins on and/or integrated into the vesicles or the membrane.
wherein the membrane protein is a trans-membrane (TM) protein, a membrane associated protein or a membrane spanning protein.

As outlined above, preferably the expression reaction is carried out in a micro-fluidic reaction chamber having dimensions and geometry to provide for a surface (A) by volume (V) ratio (A/V) of the fluid in the reaction chamber of at least 1 mm⁻¹, preferably at least 3 mm⁻¹, more preferably at least 5 mm⁻¹ or at least 10 mm⁻¹.

In another preferred embodiment the at least one micro-fluidic reaction chamber has a cross sectional area over its entire length of 2×10⁻³ μm² to 3×10⁶ μm², preferably 1 μm² to 1×10⁶ μm², more preferably 1×10³ μm² to 0.5×10⁶ μm².

In yet another preferred embodiment of the process of the present invention the membrane proteins are selected from trans-membrane proteins, membrane associated proteins, and membrane spanning proteins, preferably are selected from the group consisting of G-protein coupled receptors, neurotransmitter receptors, kinases, porins, ABC transporters, ion transporters, acetylcholin receptors and cell adhesion receptors.

The in vitro transcription and translation requires the addition of nucleic acid to be transcribed, whereby preferably the nucleic acid coding for the membrane proteins is added as cDNA. The cDNA can be derived from a commercial or customized cDNA library.

An essential component of the present invention is the use of an in vitro transcription and translation system, which is a cell-free expression system. By the cell-free expression system a nucleic acid coding for the desired membrane protein, optionally also coding for a tag, is transcribed and translated and, thus, the desired membrane protein is formed in situ and then immediately incorporated into the synthetic membrane. Such a cell-free expression system is described e.g. in U.S. Pat. No. 5,324,637. Preferably, a eukaryotic cell-free extract is used as an expression system. Such expression systems are commercially available, e.g. as TNT(R) coupled transcription/translation system from Promega Corporation. However, it is also possible to use a prokaryotic cell-free expression system (e.g. RTS 100 E. coli hy Kit™ from Roche Applied Science) or an archaic cell-free expression system (e.g. Sarma; E. M. Fleischmann, Cold Spring Harbour Press, ISBN 0-87969-438-6, Protocol 18, p. 133).

As described above, surprisingly, it was found that expression of the membrane proteins with a cell-free expression system, preferably a eukaryotic cell-free expression system, in the presence of a synthetic membrane leads to the integration and incorporation of the membrane proteins into the synthetic membrane, whereby the membrane proteins are in a functionally active form. Thus, the membranes obtainable according to the present invention are highly suitable as assay systems in research, in particular, for investigation of interactions between membrane proteins such as receptors and their ligands. The invention, therefore, also relates to a synthetic membrane having incorporated therein a membrane protein, which synthetic membrane is obtainable by the process as described herein. The weight ratio of membrane proteins incorporated into membrane lipids is preferably 1:1 to 1:10000, in particular, 1:100 to 1:1000.

The inventive membranes having incorporated therein functionally active membrane proteins can be used as assay systems for determining the function and/or structure of membrane proteins and, in particular, for investigation of receptor/ligand interactions. However, they can also be used in sensor technology, e.g. as an odorant receptor. Further uses are warfare applications, detection of biotoxic, e.g. anthrax, toxic or explosive material, ion sensors, drugs sensors or amino acid sensors.

The present invention will now be further explained and described by way of the accompanying figures and the following examples. However, the figures and the examples are not intended to restrict the invention.

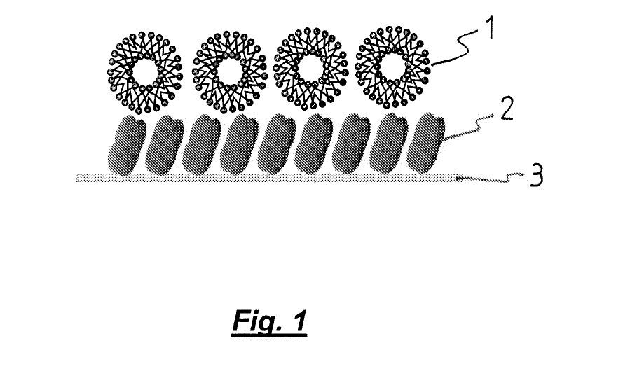

FIG. 1 shows lipid vesicles (1) bound to a gold electrode base plate (3) through hydrophilic polymer spacer molecules (2), such as cationic bovine serum albumin (cBSA) according to the present invention.

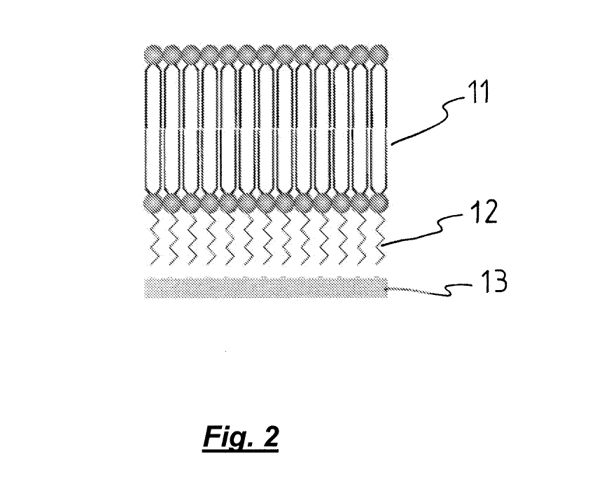

FIG. 2 shows a lipid membrane (11) bound to a gold electrode base plate (13) through spacer molecules (12) according to the present invention.

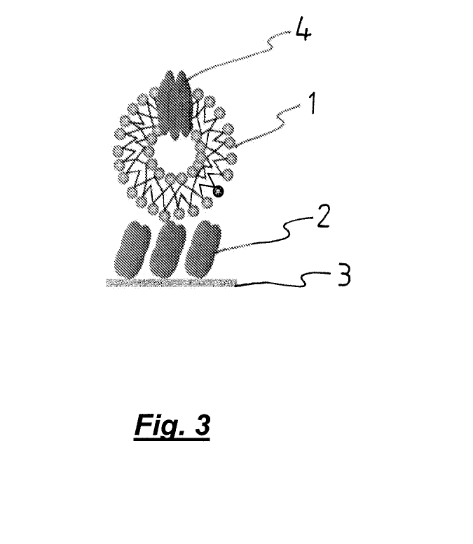

FIG. 3 shows a lipid vesicle according to FIG. 1 having an ion channel (4) (membrane protein) in vitro synthesised into the lipid bilayer of the vesicular membrane.

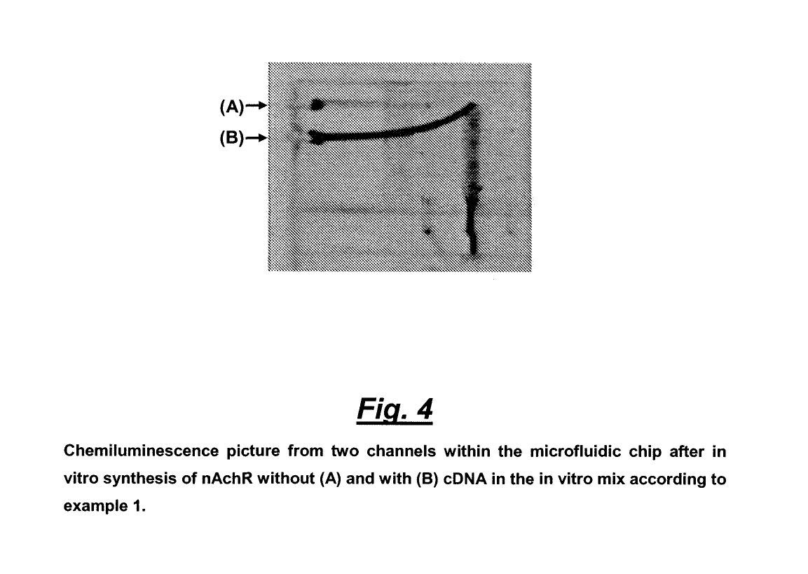

FIG. 4 shows a chemiluminescence picture from two channels within the microfluidic chip after in vitro synthesis of nAchR without (left) and with (right) cDNA in the in vitro mix of Example 1)

### EXAMPLES

### Example 1

### Assembly and Functionalisation of the (Biomimetic) Lipid Environment

### (Impedance Data and Antibody Labelling)

A gold electrode array for a micro-fluidic chip was treated with acetone and isopropanol to remove the protecting lacquer. Then it was treated for 5 min in an argon plasma (0.19 mbar, 310 W) and directly guided into the micro-fluidic reaction chambers of the micro-fluidic chip. The micro-fluidic reaction chambers had a rectangular geometry and dimensions of 0.2 mm height, 2 mm width, and 20 mm length, thus providing for a surface (A) by volume (V) ratio (A/V) in the reaction chamber of 5 mm⁻¹ and a cross sectional area of 0.2 mm height×2 mm width=0.4 mm².

All filling steps were conducted with a peristaltic pump at a flow rate of 100 μl/min. All impedance scan measurements were taken in the frequency range between 0.1 MHz and 5 MHz (logarithmic distribution of 30 measurement points) with a DC potential of 0 V and an AC amplitude of 10 mV.

The channels of the micro-fluidic chip were rinsed with phosphate buffered saline (PBS) and an impedance scan was measured. After the scans the channels were filled with 0.01 mg/ml cationic bovine serum albumin (cBSA) in PBS and incubated for 2 h at room temperature, then rinsed with PBS for 5 min, followed directly by the next impedance scan.

For the vesicle preparation 2 μl of a 3-sn-phosphytidylcholine (PC) solution (10% in chloroform) were dried in a glass tube under a nitrogen stream, redissolved in 1 ml PBS and sonicated at +50° C. for 10 min. For a more homogeneous size distribution the solution was extruded with a commercial extruder through a polycarbonate membrane (pore size 50 nm) for 21 passages. The vesicle solution was filled into the channels of the micro-fluidic chip and incubated at +4° C. over night (ca. 16 h). The next day the channels were rinsed with PBS for 2 min followed by an impedance scan.

For the next step the in vitro synthesis (IVS) mixture in an E. coli extract from Promega was prepared in a 1.5 ml Eppendorf tube with the following composition where the cDNA is coding for the α7 subunit of the nicotinic Acetylcholine Receptor (nAchR) cloned into the plasmid pTNT with a VSV (Vesicular stomatitis virus)-tag at the N-terminus of the protein.

| | Positive reaction | negative control |
|---|---|---|
| S30 PremixPlus | 20 μl | 20 μl |
| T7/S30 Extract | 18 μl | 18 μl |
| Nuclease free water | 9 μl | 12 μl |
| cDNA (c = 0.43 μg/μl) | 3 μl | — |

Then each IVS mixture was filled into two different micro-fluidic reaction chambers and incubated for 1.5 h at 37° C. in an incubator. Afterwards both micro-fluidic reaction chambers were rinsed with PBS for 2 min.

### Antibody Labelling

### (Chemiluminescence Image)

The antibody detection was conducted with a commercially available kit for chemiluminescence detections from Invitrogen. All filling steps were conducted with a peristaltic pump at a flow rate of 100 μl/min.

The different solutions were prepared according to the manufacturer's protocol:
Blocking solution: 7 ml ultrapure water
   2 ml Blocker Diluent Part A
   1 ml Blocker Diluent Part B
Washing solution: 7.5 ml ultrapure water
   0.5 ml wash solution
1^{st} Antibody solution: 180 μl PBS
   20 μl 1^{st} Antibody (Anti-VSV from mouse)

For each incubation step the device was placed on a shaker set at 85 rpm.

Each channel was rinsed with blocking solution for 2 min and then incubated for 30 min; rinsed with ultrapure water for 2 min; filled with 1st antibody solution (half of the batch per channel) and incubated for 2 h; rinsed with washing solution for 2 min; filled with 2^{nd} antibody solution (anti-mouse) and incubated for 30 min; rinsed with washing solution for 2 min; filled with chemiluminescent substrate and incubated for 5 min.

The 2^{nd} antibody solution is an alkaline phosphatase-conjugated IgG that can be detected using a gel-imager in chemiluminescence mode. The sample was exposed continuously and every 2 min a picture was taken. The result presented in FIG. 4 was obtained after 24 min.

### Example 2

### Activity Measurements

All data was measured with a “Surfe2r One” from IonGate Bioscience GmbH, Germany. The sensor consists of a 7 mm² gold surface within an open-top device where the solution can be exchanged rapidly via a sophisticated fluidic system.

On top of a self-assembled hybrid layer a lipid bilayer was attached. Two different approaches for this bilayer were used—either a solution of small unilamellar vesicles or a preparation of membrane fragments was used.

Lipid vesicles were made of a 1:1 ratio of soybean phosphatidylcholine and cholesterol to a concentration of 2 mg/ml in PBS. Small unilamellar vesicles were formed by 2×10 sonication pulses (0.5 s, 30% amplitude) and extrusion with a commercial extruder through a polycarbonate membrane (pore size 50 nm) for 21 passages.

For other samples membrane fragments of PepT-rich CHO cells were used where PepT is a carrier for dipeptides.

The clean sensors were incubated in a Sensor Prep A solution (2 mM Octadecanthiol (ODT) in isoporopanol from IonGate) for 24 h. The sensor was rinsed with ultrapure water and dried under nitrogen. 2 μl Sensor Prep B (Diphytanoylphosphatidylcholine in decane from IonGate) were applied to the surface and 48 μl of in vitro synthesis (IVS) mixture were added immediately.

Therefore a rabbit reticulocyte lysate IVS mixture from Promega was used and prepared in a tube without the DNA according to the following:

| | |
|---|---|
| 40 μl | TnT ® T7 Quick Master Mix |
| 2 μl | Methionine (1 mM) |
| 6 μl | Vesicle solution/respectively membrane fragment solution |

The buffer solution was removed from the sensor surface ad the IVS mixture was added. The sensors were centrifuged at 2500 g for 45 min. For each sensor types (vesicles or membranes) a positive reaction (with cDNA) and a negative control (without cDNA) was prepared. For the positive reaction 2 μl cDNA with a concentration of c=0.98 μg/μl (coding for the α7 subunit of the nicotinic Acetylcholine Receptor (nAchR) cloned into the plasmid pTNT) and for the negative control 2 μl nuclease free water were added under sterile conditions. The sensors were incubated at 32° C. for 85 min.

The measurement concept is based on the rapid exchange of buffers to create an ion gradient. Therefore different buffers were used.

| | | |
|---|---|---|
| Buffer C: | 100M | NaCl (Sodium chloride) |
| | 3M | EGTA (ethylene glycol tetraacetic acid) |
| | 3M | EDTA (ethylenediaminetetraacetic acid) |
| | 30M | Tris (tris(hydroxymethyl)aminomethane |
| Buffer B: | 100M | NMG (N-methyl-D-glucamine) |
| | 3M | EGTA |
| | 3M | EDTA |
| | 30M | Tris |
| Buffer A: | 100M | NMG |
| | 3M | EGTA |
| | 3M | EDTA |
| | 30M | Tris |
| | 100 μM | Carbamoylcholine chloride |

Carbamoylcholine chloride is a specific ligand that binds to and activates the nAchR.

For measuring the sensors were mounted into the instrument and rinsed with buffer C at a flow rate of 220 μl/min. The sensor was incubated for up to 10 min to allow homogeneous distribution of ions.

All exchanges of the buffer during the measurement were at a flow rate of 300 μl/min.

The measurement starts in Buffer C which is replaced after 1 s by Buffer B for 5 s (establishment of the ion gradient). After these 5 s buffer B is replaced by Buffer A (activation) for 1 s followed directly by Buffer B for 1 s and ends in Buffer C measured for 3 s.

So the sequence is:
C(1 s)-B(5 s)-A(1 s)-B(1 s)-C(3 s)

The sensors with the positive reaction were treated with an inhibitor. For the inhibition the specific antagonist α-Bungarotoxin (BTX)m, a snake venom—was used. The sensor was kept in Buffer C and BTX was added to a final concentration of c=100 nM and incubated for approximately 30 min. The same concentration of BTX was added to the buffer reservoirs A, B and C as well. After the incubation the sensors were rinsed with Buffer C+BTX at 220 μl/min and then measured with the same sequence mentioned above.

After the inhibition the sensors were rinsed with Buffer C without antagonist to wash out the BTX and then measured again in BTX-free buffer.

The measurement showed that addition of carbamoylcholinechloride results in a signal which could not be generated the same way after incubation with the inhibitor α-BTX. The negative control without DNA showed no reaction to any activation. The results are strong indications for an actual successful incorporation of a functional nAchR. The results and the form of the signals which allows to distinguish between inward and outward currents leads to the assumption that the attachment took place vectorially and in this case with the extracellular side of the protein located on top of the sensor.

1. A system for the in vitro transcription and translation of membrane proteins comprising:
i) a micro-fluidic chip having at least one micro-fluidic reaction chamber and micro-fluidic channels to allow fluid to flow through the chip and into and from the at least one reaction chamber,
ii) the at least one micro-fluidic reaction chamber being provided with at least one electrode base plate of conductive or semi-conductive material, and
iii) lipid vesicles or a lipid membrane being bound or tethered to the at least one electrode base plate either directly or through spacer molecules.

2. The system of claim 1, wherein the at least one micro-fluidic reaction chamber has dimensions and geometry to provide for a surface (A) by volume (V) ratio (A/V) of the fluid in the reaction chamber of at least 1 mm⁻¹, preferably at least 3 mm⁻¹, more preferably at least 5 mm⁻¹ or at least 10 mm⁻¹.

3. The system of claim 1, wherein the at least one micro-fluidic reaction chamber has a cross sectional area over its entire length of 2×10⁻³ μm² to 3×10⁶ μm², preferably 1 μm² to 1×10⁶ μm², more preferably 1×10³ μm² to 0.5×10⁶ μm².

4. The system of claim 1, wherein the at least one electrode base plate consists of or has a surface consisting of a metal, metal oxide, polymeric materials, glass, field effect transistors, indium tin oxide (ITO), preferably the electrode base plate consists of gold.

5. The system of claim 1, wherein the lipid vesicles or the lipid membrane are/is bound or tethered to the at least one electrode base plate through spacer molecules, preferably the spacer molecules being selected from the group consisting of human serum albumine molecules (HAS), bovine serum albumine molecules (BSA) or cationic bovine serum albumine molecules (cBSA), poly-peptide or oligo-peptide molecules, PEG, sugar molecules, silane molecules, silane/thiol molecules, or polymer molecules.

6. The system of claim 1, wherein the lipid vesicles or the lipid membrane are/is synthetic or comprise/s natural membrane components, synthetically produced lipids, phospholipids, preferably 1,2-Dimyristoyl-sn-glycero-3-phosphoethanolamine (DMPE) or phosphatidylcholine.

7. The system of claim 1, wherein the lipid vesicles or the lipid membrane comprise/s a lipid bilayer.

8. A process for the in vitro transcription and translation of membrane proteins comprising:
i) providing a system according to claim 1,
ii) applying a cell-free expression system and a nucleic acid coding for the membrane proteins to the lipid vesicles or the lipid membrane in the at least one micro-fluidic reaction chamber, and
iii) expression of the membrane proteins on and/or integrated into the vesicles or the membrane,
wherein the membrane protein is a TM protein, a membrane associated protein or a membrane spanning protein.

9. The process of claim 8, wherein the expression reaction is carried out in a micro-fluidic reaction chamber having dimensions and geometry to provide for a surface (A) by volume (V) ratio (A/V) of the fluid in the reaction chamber of at least 1 mm⁻¹, preferably at least 3 mm⁻¹, more preferably at least 5 mm⁻¹ or at least 10 mm⁻¹ and/or the at least one micro-fluidic reaction chamber having a cross sectional area over its entire length of 2×10⁻³ μm² to 3×10⁶ μm², preferably 1 μm² to 1×10⁶ μm², more preferably 1×10³ μm² to 0.5×10⁶ μm².

10. The process of claim 8, wherein the membrane proteins are selected from trans-membrane proteins, membrane associated proteins, and membrane spanning proteins, preferably are selected from the group consisting of G-protein coupled receptors, neurotransmitter receptors, kinases, porins, ABC transporters, ion transporters, acetylcholin receptors and cell adhesion receptors.

11. The process of claim 8, wherein the nucleic acid coding for the membrane proteins is added as cDNA.
